(19) United States
(12) Patent Application Publication
(10) Pub. No.: US 2009/0191138 A1
(43) Pub. Date: Jul. 30, 2009

(54) NOVEL TOPICAL FORMULATIONS FOR IMPROVING THE APPEARANCE OF NAILS

(75) Inventor: Frederick J. Dechow, Kirkland, WA (US)

Correspondence Address: CONNOLLY BOVE LODGE & HUTZ LLP, 1875 EYE STREET, N.W., SUITE 1100, WASHINGTON, DC, 20006, US

(73) Assignee: MediQuest Therapeutics, Inc., Bothell, WA (US)

(21) Appl. No.: 12/022,833

(22) Filed: Jan. 30, 2008

(51) Int. Cl. A61K 8/18

(52) U.S. Cl. 424/61

(57) ABSTRACT

A method is provided for improving the appearance of nails of a patient wherein a composition is topically applying to the patient's nail(s) exhibiting symptoms of at least one condition selected from the group consisting of onycholysis, onychoschizia or onychorrhexis and wherein the composition contains one or more biocompatible organic solvents, a polar lipid, a surfactant, water, urea and a thickener wherein the organic solvents include an ester and/or a dihydric and/or polyhydric alcohol is provided.

### TECHNICAL FIELD

This disclosure relates to a composition useful in the improvement of appearance of nails. It has been discovered according the present disclosure that a topical microemulsion is able to improve the appearance of fingernails and toenails that are showing the symptoms of onycholysis, onychoschizia and/or onychorrhexis.

### BACKGROUND

Onycholysis is a nail disorder frequently encountered by dermatologists. Onycholysis is characterized by a spontaneous separation of the nail plate starting at the distal free margin and progressing proximally. The nail plate is separated from the underlying and/or lateral supporting structures. Less often, separation of the nail plate begins at the proximal nail and extends to the free edge, which is seen most often in psoriasis of the nails (termed onychomadesis). Rare cases are confined to the nail's lateral borders.

Nails with onycholysis usually are smooth, firm, and without inflammatory reaction. It is not a disease of the nail matrix, but nail discoloration may appear underneath the nail as a result of secondary condition. Treating primary and secondary factors that exacerbate the condition is important. Left untreated, severe cases of onycholysis may result in nail bed scarring.

Endogenous, exogenous, hereditary, and idiopathic factors can cause onycholysis. Contact irritants, trauma, and moisture are the most common causes of onycholysis, but other associations exist. Endogenous factors occur from systemic diseases and conditions such as: Amyloid and multiple myeloma, Anemia (iron deficient), Bronchiectasis, Diabetes mellitus, Erythropoietic porphyria, Histiocytosis X, Hyperthyroidism, Hypothyroidism, Ischemia (peripheral, impaired circulation), Leprosy, Lupus erythematosus, Neuritis, Pellagra, Pemphigus vulgaris, Pleural effusion, Porphyria cutanea tarda, Pregnancy, Psoriatic arthritis, Reiter syndrome, Sarcoidosis, Scleroderma, Shell nail syndrome, Syphilis, and Yellow nail syndrome. It may also arise from dermatologic diseases such as: Psoriasis, Lichen planus, Dermatitis, Hyperhidrosis, Pachonychia congenital, Congenital ectodermal defect, Pemphigus vegetans, Lichen striatus, Atopic dermatitis, and Congenital abnormalities of the nail.

Exogenous factors include microbial factors, such as Dermatophytosis (ie, Trichophyton rubrum, Trichophyton mentagrophytes infection), Yeast (Candida infection), Bacteria (Pseudomonas infection), or Virus (herpes simplex infection).

Exogenous factors may also be attributed to non-microbial factors (which may be encountered at the job site, i.e., as occupational onycholysis) and subdivided into mechanical (mechanical force (trauma), repetitive minor trauma, or maceration) or chemical (allergic contact dermatitis from various nail cosmetics (methyl methacrylate monomer, formaldehyde 1-2%, nail base coat/hardeners, polymerized 2-ethylcyanoacrylate adhesive used in artificial nails, nail lacquer), gasoline, paint removers, dicyanodiamide, thioglycolate, solvents, and hydroxylamine sulphate in color developer or irritant contact dermatitis from prolonged immersion of nails in water, sugar onycholysis in confectioners/bakers, and exposure to highly destructive toxins (e.g, hydrofluoric acid).

Nail splitting, known medically as onychoschizia, if the splitting is horizontal, or onychorrhexis, if the splitting is vertical, is a condition that causes splitting within the nail plate. The two conditions are also called “brittle nail syndrome.”

This syndrome affects nearly 20% of the population, and is seen most frequently in women and older individuals. Frequent wetting and drying of the hands is the most common cause of nail splitting and is therefore common among house cleaners, nurses, and hairdressers. Nail splitting may also be caused by nail cosmetics, nail procedures, exposures to various chemicals, such as alkalis, acids, thioglycolates, solvents, salt and sugar solutions. Injury to the nail may also be a factor in the development of nail splitting. Skin diseases, such as psoriasis and Sjögren's syndrome, endocrine diseases, malnutrition, and oral medications made from vitamin A may also be causative factors in nail splitting.

### SUMMARY

In a clinical trial to test formulations containing anti-fungal agents, it has been seen that the vehicle formulation itself is able to improve the appearance of onycholysis of patients. This was completely unexpected, since there are no known effective topical therapies to treat onycholysis.

Nails which have shown splitting have also been treated with similar formulations to those shown to treat onycholysis. In each case the nails splits have improved upon daily application of the formulation, after hand washing, for as few as five days.

The present disclosure also relates to a method of improving the appearance of fingernails or toenails displaying onycholysis, onychoschizia or onychorrhexis comprising topically applying to the nail of the human or animal certain compositions.

The composition comprises one or more biocompatible organic solvents, a polar lipid, at least one surfactant, water, urea and a thickener. The organic solvents comprise an ester and/or a dihydric and/or polyhydric alcohol. The composition comprises about 2 to about 30% by weight of the ester and/or about 2 to about 20% by weight of the dihydric and/or polyhydric alcohol.

Other objectives and advantages of the present disclosure will become readily apparent to those skilled in this art from the following detailed description, wherein it is shown and described only the preferred embodiments, simply by way of illustration of the best mode contemplated of carrying out the disclosure. As will be realized, the disclosure is capable of other and different embodiments, and its several details are capable of modifications in various obvious respects, without departing from the disclosure. Accordingly, the description is to be regarded as illustrative in nature and not as restrictive.

### BEST AND VARIOUS MODES

By “topical administration”, as used herein, is meant directly laying or spreading upon epidermal tissue, especially finger nails and toenails, including the skin areas around those nails.

By the term “comprising”, as used herein, is meant that various other compatible components such as inert ingredients, occlusive agents, and cosmetic vehicles, cosmetics and/or medicaments can be conjointly employed in the compositions and methods of this invention. The term “comprising” thus encompasses and includes the more restrictive terms “consisting of” and “consisting essentially of” which characterize the use of the essential ingredients in the manner disclosed herein.

By “afflicted sites”, as used herein, is meant a localized area of the unsightly nails, and the immediately surrounding area.

By “application sites”, as used herein, is meant a site suitable for application via a mechanical release device or dressing, e.g., at the distal end of the nails, on the top of the nails, at the proximal area of the nails, etc.

By “substantially free”, as used herein, is meant that the compositions of the present invention contain less than about 10%, preferably less than 3.5%, more preferably less than about 1%, and most preferably less than about 0.5%, of any specific compound, or member of the group of compounds, described by this term.

As used herein, all percentages and ratios are by weight of the total composition unless otherwise specified.

The compositions employed in this disclosure comprise a polar lipid, such as lecithin or phosphotidylcholine, and one or more biocompatible organic solvents, one chosen from the group of esters and/or one chosen from the group of liquid dihydric and polyhydric alcohols, a surfactant, water, and urea, at a pH of about 5 to about 8.5 and preferably of about 6 to about 7.5. The compositions of this disclosure may additionally contain other optional components that reduce skin irritation, enhance physical stability or enhance their cosmetic appeal or acceptability, e.g, emollients, thickeners, pigments, fragrances, perfumes, preservatives and the like. The compositions of this disclosure may or may not contain a cosmetic agent and/or pharmaceutically-active agent capable of producing or possessing local activity, with the composition of this disclosure as the carrier.

Typical polar lipids employed are lecithin and phosphotidylcholine. Preferably, the lecithin or phosphatidylcholine is of a high quality, pharmaceutical grade. Appropriate lecithin and phosphatidylcholine maybe obtained as commercially available soya lecithin or soya phosphatidylcholine. Preferably, soya lecithin is used in the composition of this disclosure.

The biocompatible organic ester solvents may be any non-toxic ester in which the polar lipid and urea are soluble. Typically the esters are fatty mono esters having a structure, obtainable by replacing the active hydrogen of a fatty acid having 4 to 22 carbon atoms and more typically having 8 to 18 carbon atoms by the alkyl group of a monohydric alcohol, a particular example being 12 carbon atoms. The fatty acid can be saturated or unsaturated and more typically is saturated. The monohydric alcohol typically contains 2 to 8 carbon atoms and more typically 2 to 5 carbon atoms, a particular example being 3 carbon atoms.

Acceptable esters for this purpose include, but are not limited to isopropyl esters. Preferably, the ester is isopropyl myristate or isopropyl palmitate, with isopropyl myristate being particularly preferred.

The biocompatible organic dihydric and polyhydric alcohol solvents may be any non-toxic dihydric alcohol or polyhydric alcohol in which the polar lipid and urea are soluble. Acceptable dihydric and polyhydric alcohols for this purpose include, but are not limited to di- and tri-alcohol alkanes. Typically the alcohols contain 3 to 8 carbon atoms and more typically 3 to 5 carbon atoms and are saturated alcohols. Preferably, the polyalcohol is propylene glycol or glycerol, with propylene glycol being particularly preferred.

The compositions of the present disclosure typically contain about 2 to 30% by weight and more typically 4 to 10% by weight of the ester and/or about 2 to about 20% by weight and more typically 2 to about 10% weight of the dihydric/polyhydric alcohol.

In preparing the composition of this disclosure, the polar lipid is typically dissolved in the organic ester solvent and/or dihydric or polyhydric alcohol solvent at mass ratios from about 5:1 to about 1:5 polar lipid:solvent. Preferably, the polar lipid and organic ester solvent and/or polyalcohol solvent are mixed in equal mass ratios. Thus, in one embodiment of the disclosure, soya lecithin, isopropyl myristate, and propylene glycol are mixed in equal mass ratios and mixed until the lecithin is evenly distributed. This is referred to as the solvent-polar lipid mixture.

A surfactant is included in the formulation typically at a concentration of about 0.5% to about 20% of the final composition mass. In the formulation including a polycationic active agent, it has been found, according to this disclosure that non-ionic or cationic surfactants are preferred. In the case of other or no active ingredients, on the other hand, anionic, cationic or non-ionic surfactants are quite acceptable. Preferably, the surfactant is one which is compatible with administration in vivo without elicitation of undesirable side effects. One preferred surfactant is docusate sodium and its more water soluble form, docusate sodium benzoate. Other appropriate ionic or non-ionic surfactants, such as polysorbate 80, Tween 80, poloxamer, ibuprofen, docusate calcium, tetradecyltrimethylammonium bromide, pentaoxyethylene glycol monododecyl ether, or triethanolamine laureth sulfate. Once the surfactant is thoroughly dispersed in the solvent-polar lipid mixture, a cosmetic or pharmaceutically active compound, if desired, may be added and dissolved. Examples of such active compounds include anti-proliferative/anti-inflammatory/anti-microbial compound that can ameliorate other contributors to the unsightly appearance of fingernails and toenails or decrease the response time of the compositions. However, compositions of this disclosure do not require and are preferably free of such active agents.

After addition of a cosmetic or pharmaceutically active compound, if used, an amount of urea, preferably as a thickened aqueous solution, can be added to the surfactant-solvent-polar lipid mixture. The urea is typically added so that the urea concentration about 1% to about 20%, more typically about 1% to about 15% and even more typically about 5% and 10% by mass of the final composition mass.

The thickener is selected from common National Formulary thickening agents including, but not limited to appropriate polymer weights of polyethylene glycol, polyvinylpyrrolidone, carbomer, alginates, gums and methylcellulose. The amount of thickener is typically about 0.01 to about 5% by weight and more typically about 0.05% to about 5%.

Thus in a specific example, about 5 grams of a 10% aqueous solution of urea, containing 0.9% Carbomer 974P, is added to about 100 grams of the surfactant-solvent-polar lipid mixture. In any event, this is a choice readily made by those skilled in the art, once aware of the present disclosure, depending on the particular formulation being prepared.

Upon formulation of the above described composition, the pH is adjusted to a typical pH of about 5 to about 8.5 and more typically to a 6 to 7.5. This can be accomplished, for example, by addition of aqueous sodium hydroxide, as the compositions initially tend to have an acid pH. However, if the pharmaceutically active agent tends to produce very alkaline solutions, addition of acid to reduce the pH would be desirable. This can be accomplished by addition of citric acid or a biological buffer such as sodium carbonate or potassium phosphate. With the composition in a pH range of about 5.0 to 8.5, the formulation thickens and forms a stable microemulsion for topical administration.

By routine experimentation, using the recited elements of this composition, those skilled in the art, once aware of the present disclosure, will be able to make specific microemulsions that may also include active ingredient or combination thereof for a wide variety of anti-inflammatory or antimicrobial applications. In addition, it is understood that the compositions can contain auxiliary agents including those conventionally known and/or used in this art such as, but not limited to, preservatives and fragrances.

For ease of preparation, it is convenient to prepare a first gel composition, referred to herein as “MQX-GEL”, which can be used to add to other components in the formulation of a final composition for topical administration. There are several possible formulations of the MQX-GEL. For example, a MQX-GEL may be prepared by mixing lecithin organogel (L.O.), as a 1:1:1 (m/m) mixture of lecithin, isopropyl myristate and propylene glycol, with LID oil (a 1:1 [m/m] mixture of L.O. and docusate sodium), dissolving additional surfactant and/or docusate sodium powder into this mixture, and then adding thickened aqueous urea.

In one embodiment of the MQX-GEL formulation, the final concentrations are: L.O.=30%; docusate sodium=9%; urea=5%; thickener=1%; and water=55%. These ratios may easily be varied such that the final amounts of each component are as follows: L.O.=15-50%; docusate sodium and/or another surfactant=3-20%; urea=1-15%; thickener=0.5-5%; and water=40-65%. The solubilized active ingredients may then be added to MQX-GEL. Excipients which may be useful in solubilizing an active ingredient, if used, include L.O., propylene glycol, isopropyl myristate, limonene, peppermint oil, glycerin, and/or polyethylene glycol. A homogenous mixture is then made by carefully blending the various components.

Once the formulations described above have been prepared, use of the formulations is a simple matter of applying the formulation to affected areas where cutaneous delivery is desired. Thus, in the case of split nails, formulations as described above are rubbed over the affected nail area of the fingers or the toes. In use of formulations prepared according to this invention, the normal appearance of non-split nails has been restored within five days with daily application. Treatment is repeated as symptoms reappear.

Likewise, in the case of onycholysis, formulations as described above are rubbed over the affected nail area of the fingers or the toes. In use of formulations prepared according to this invention, the normal appearance of nails has been restored within four months with daily application. Treatment is repeated as symptoms reappear.

It is contemplated that the compositions of this invention are applied topically as frequently as required as long as local reactions do not become a problem.

While the foregoing description generally describes how to carry out the present disclosure, the following examples are provided to more specifically point out how to practice the invention. However, it should be clearly understood that the scope of this invention, as defined by the claims appended hereto, is not to be limited to the specifics of the following examples. Further, it should be understood that, in the specific compositions described and claimed, the percentages of ingredients could be within at least a 10% different amount while still achieving an objective equivalent to the specifically disclosed compositions.

In general the formulation has the following range of ingredients:

Formulation
Ranges

| | Low | High |
|---|---|---|
| Purified Water, USP | 30 | 65 |
| Urea, USP | 1 | 20 |
| Thickeners (examples below) | 0.01 | 5 |
| Carbopol 974P, NF | 0.01 | 5 |
| Methocellulose (var. grades) | 0.01 | 5 |
| Polyethylene glycol | 0.01 | 5 |
| Povidone | 0.01 | 5 |
| Isopropyl Ester | 2 | 30 |
| Isopropyl Myristate, NF | 2 | 30 |
| Polyhydric Alcohol | 0.5 | 20 |
| Propylene Glycol, USP | 0.5 | 20 |
| Polar Lipid | 5 | 30 |
| Lecithin, NF | 5 | 30 |
| Surfactants | 0.5 | 20 |
| Docusate Sodium, USP | 0.5 | 20 |
| Polysorbate 80, NF | 0.5 | 20 |
| pH Adjusted to, using: | 5 | 8.5 |
| Sodium Hydroxide, NF (1N) | 0 | QS |
| Trolamine, NF | 0 | QS |

The specific formulations tested were:

Formulation

| | A | B |
|---|---|---|
| Purified Water, USP | 60.4 | 60.4 |
| Urea, USP | 10 | 10 |
| Carbopol 934, NF | 0.05 | 0.00 |
| Carbopol 974P, NF | 0.00 | 0.05 |
| Trolamine, NF | 0.07 | 0.07 |
| Isopropyl Myristate, NF | 6.53 | 6.53 |
| Methocellulose, USP | 0 | 0 |
| Lecithin, NF | 7.31 | 7.31 |
| Docusate Sodium, USP | 14.36 | 14.36 |
| Propylene Glycol, USP | 0.98 | 0.98 |
| Polysorbate 80, NF | 0.3 | 0.3 |
| Sodium Hydroxide, NF (1N) | 0 | 0 |
| | 100 | 100 |

Additional formulations that have been studied are:

Formulation

| | C | D |
|---|---|---|
| Purified Water, USP | 50.68 | 50.68 |
| Urea, USP | 10 | 10 |
| Carbopol 934, NF | 0 | 0 |
| Carbopol 974P, NF | 0.07 | 0.05 |
| Trolamine, NF | 0.06 | 0.06 |
| Isopropyl Myristate, NF | 8.7 | 8.7 |
| Methocellulose, USP | 0.03 | 0.03 |
| Lecithin, NF | 9.75 | 9.75 |
| Docusate Sodium, USP | 19.14 | 19.14 |
| Propylene Glycol, USP | 9 | 12 |
| Polysorbate 80, NF | 0.4 | 0.4 |
| Sodium Hydroxide, NF (1N) | 0.48 | 0.48 |
| | 100 | 100 |

Formulation

| | E | F |
|---|---|---|
| Purified Water, USP | 62.25 | 48.4 |
| Urea, USP | 10 | 10.68 |
| Carbopol 934, NF | 0 | 0 |
| Carbopol 974P, NF | 0.05 | 0.07 |
| Trolamine, NF | 0.07 | 0.07 |
| Isopropyl Myristate, NF | 6.09 | 8.7 |
| Methocellulose, USP | 0.05 | 0.05 |
| Lecithin, NF | 6.83 | 9.75 |
| Docusate Sodium, USP | 13.4 | 19.14 |
| Propylene Glycol, USP | 0.98 | 0.98 |
| Polysorbate 80, NF | 0.28 | 0.4 |
| Sodium Hydroxide, NF (1N) | 0.3 | 0 |
| | 100 | 100 |

The following non-limiting examples are presented to further illustrate the present disclosure:

### EXAMPLE 1

### Preparation of MQX-GEL

| | 500 gm | |
|---|---|---|
| LID Oil* | 50 | gm |
| Lecithin organogel** (L.O.) | 100 | gm |
| Docusate sodium powder | 50 | gm |
| Urea | 50 | gm |
| Thickener | 5 | gm |
| Distilled water | 245 | ml |

*LID oil is a 1:1 mixture of lecithin organogel:docusate sodium on a mass basis.
**L.O. is a 1:1:1 mixture of lecithin, isopropyl myristate and propylene glycol.1.
1. The LID was added to L.O. and heated.
2. Docusate sodium powder was added, and the mixture was stirred until smooth.
3. Thickener and urea were completely dissolved in water, heated, and added to step 2 with stirring.
4. pH was adjusted to between 6.5 to 6.9.

MQX-GEL may just as easily be prepared as follows:

| | 1000 gm | |
|---|---|---|
| L.O. | 250 | gm |
| Docusate sodium benzoate powder | 150 | gm |
| Urea | 100 | gm |
| Thickener | 10 | gm |
| Distilled water | 490 | ml |

The L.O. was heated and the docusate sodium benzoate powder was stirred into the heated L.O. until a smooth solution is prepared. The water: was heated and the thickener and urea were dissolved into the water, and the thickened urea solution was then thoroughly mixed with the docusate sodium containing solution of L.O. The result was a consistent, transparent, amber colored gel with a pH of about 6.0.

A further method of making MQX-GEL is as follows:

| | 1000 gm | |
|---|---|---|
| L.O. | 100 | gm |
| LID | 300 | gm |
| Urea | 100 | gm |
| Thickener | 10 | gm |
| Distilled water | 490 | gm |

The LID and L.O. were mixed well and a heated solution of water, the thickener and the urea was prepared and added to the LID-L.O. solution. The result was a consistent, transparent, amber colored gel with a pH of about 6.0.

The same method of combining the ingredients is used as described in example 2.

MQX-GEL can also be prepared with other ratios of the three constituents of the lecithin organogel. In the following example, the ratio of lecithin organogel (L.O. #2), is a 1:0.9:0.1 (m/m/m) mixture of lecithin, isopropyl myristate and propylene glycol, with LID oil (a 1:1 [m/m] mixture of L.O.#2 and docusate sodium), dissolving additional surfactant and/or docusate sodium powder into this mixture, and then adding thickened aqueous urea.

In this embodiment of the MQX-GEL formulation, the final concentrations are: L.O.#2=25%; docusate sodium=10%; urea=10%; thickener=1%; and water=54%. These ratios also may easily be varied such that the final amounts of each component are as follows: L.O.#2=15-50%; docusate sodium and/or another surfactant=3-15%; urea=1-15%; thickener=0.5-5%; and water-40-65%. A solubilized active ingredients, if desired, can then be added to MQX-GEL. Excipients which may be useful in solubilizing an active ingredient, if used, include L.O.#2, propylene glycol, isopropyl myristate, peppermint oil, glycerin, and/or polyethylene glycol. A homogenous mixture is then made by carefully blending the various components.

### EXAMPLE 3

### Preparation of Another MQX-GEL

| | 500 gm | |
|---|---|---|
| Propylene Glycol | 6.0 | gm |
| L.O. #2 | 115.0 | gm |
| Docusate Sodium | 45.0 | gm |
| Urea | 45.0 | gm |
| Carbomer 974P | 3.5 | gm |
| Methylcellulose | 4.4 | gm |
| Water, distilled | 227.1 | gm |

The same method of combining the ingredients is used as in example 2.

### EXAMPLE 4

### Preparation of Another MQX-GEL

| | 500 gm | |
|---|---|---|
| Proplyene glycol | 3.0 | gm |
| L.O.#2 | 155.0 | gm |
| Docusate Sodium | 50.0 | gm |
| Urea | 50.0 | gm |
| Carbomer 974P | 3.5 | gm |
| Methylcellulose | 4.4 | gm |
| Water, distilled | 234.1 | gm |

The same preparation method was used in this example as in the previous one.

### EXAMPLE 5

### Preparation of Another MQX-GEL

| | 500 gm | |
|---|---|---|
| Proplyene Glycol | 1.0 | gm |
| L.O.#2 | 128.9 | gm |
| Docusate Sodium | 65.0 | gm |
| Urea | 50.0 | gm |
| Carbomer 974P | 2.6 | gm |
| Methylcellulose | 1.5 | gm |
| Water, distilled | 251.0 | gm |

1. Propylene glycol is added to L.O.#2 and stirred to obtain a clear solution.
2. Docusate sodium is added to the solution from step 1 and stirred to obtain a clear solution.
3. Urea is added to distilled water, with heating and stirring to obtain a uniform solution.
4. Carbomer 974P and Methylcellulose are added to thicken the urea-water of step 3.
5. The solution from step 2 is combined with the thickened aqueous urea from step 4 to form a uniform mixture.
6. The pH is adjusted to 6.5 with dilute aqueous NaOH to form an elegant thick microemulsion.

The foregoing description illustrates and describes the present disclosure. Additionally, the disclosure shows and describes only the preferred embodiments of the disclosure, but, as mentioned above, it is to be understood that it is capable of changes or modifications within the scope of the concept as expressed herein, commensurate with the above teachings and/or the skill or knowledge of the relevant art. The embodiments described hereinabove are further intended to explain best modes known of practicing the invention and to enable others skilled in the art to utilize the disclosure in such, or other, embodiments and with the various modifications required by the particular applications or uses disclosed herein. Accordingly, the description is not intended to limit the invention to the form disclosed herein. Also, it is intended that the appended claims be construed to include alternative embodiments.

All publications, patents and patent applications cited in this specification are herein incorporated by reference, and for any and all purposes, as if each individual publication, patent or patent application were specifically and individually indicates to be incorporated by reference. In the case of inconsistencies, the present disclosure will prevail.

1. A method for improving the appearance of nails of a patient in need thereof which comprises topically applying to the patient's nail(s) exhibiting symptoms of at least one condition selected from the group consisting of onycholysis, onychoschizia or onychorrhexis, a composition which comprises one or more biocompatible organic solvents, a polar lipid, at least one or more surfactant, water, urea and thickener; wherein the organic solvents comprise an ester and/or a dihydric alcohol and/or polyhydric alcohol; and wherein the composition comprises about 2 to about 30% of the ester and/or about 0.50% to about 20% of the dihydric alcohol and/or polyhydric alcohol and wherein said composition is free from auxiliary antimicrobial agent.

2. The method of claim 1, wherein the ester is a fatty monoester.

3. The method of claim 2, wherein the ester is obtainable by replacing the active hydrogen of a fatty acid having 4 to 22 carbon atoms by the alkyl group of a monohydric alcohol having 2 to about 8 carbon atoms.

4. The method of claim 2, wherein the ester is an isopropyl ester.

5. The method of claim 1, wherein the ester is at least one of isopropyl myristate or isopropyl palmitate.

6. The method of claim 1, wherein the ester is isopropyl myristate.

7. The method of claim 1, wherein the dihydric or polyhydric alcohol is an alkane alcohol and contains 3 to 8 carbon atoms.

8. The method of anyone of claims 1-6, wherein the alcohol is at least one of propylene glycol or glycerol.

9. The method of anyone of claims 1-6, wherein the alcohol is propylene glyco.

10. The method of claim 1, wherein the polar lipid is at least one of lecithin or phosphalidylcholine.

11. The method of claim 1, wherein at least one surfactant is selected from the group consisting of docusate sodium, docusate sodium benzoate, docusate calcium, poloxamer, ibuprofen, tetradecyltrimethylammonium bromide, pentaoxyethylene glyco monododecyl ether, and triethanolamine laureth sulfate.

12. The method according to claim 2, wherein the thickener is selected from the group of polyethylene glycol, methyl cellulose, and carbomer.

13. The method of claim 1, wherein the amount of the polar lipid is about 5 to about 30% by weight; the amount of urea is about 1 to 20% by weight; the amount of the surfactant is about 0.5 to about 20% by weight, the amount of water is about 30 to about 65% by weight, and amount of the thickener is about 0.01 to about 5% of weight.
